SEQUENCE LISTING (5 sequences)

SEQ ID NO: 1 - protein, 771 aa, Pyrococcus abyssi
  misc_feature (705)..(705): Xaa can be any naturally occurring amino acid
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPAIRRTINLPTYTLETVYEVIFGKSKEKVYAHEIAEAWETGKGLERVAKYSMEDAKVTSELGKEFFPMEAQLARLVGHPVWDVSRSSTGNLVEWFLLTKAYERNELAPNKPDEREYERRLRESYEGGYVNEPEKGLWEGIVSLDFRSLYPSIIITHNVSPDTLNRENCKEYDVAPQVGHRFCKDFPGFIPSLLGNLLEERQKIKKRMKESKDPVEKKLLDYRQRAIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLXGDGPISKRAIAIEEFDPKKHKYDAEYYIENQVLPAVERILRAFGYRKEDLRYQKTKQVGLGAWLKF

SEQ ID NO: 2 - protein, 771 aa, Pyrococcus abyssi
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVAIATLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPAIRRTINLPTYTLETVYEVIFGKSKEKVYAHEIAEAWETGKGLERVAKYSMEDAKVTSELGKEFFPMEAQLARLVGHPVWDVSRSSTGNLVEWFLLTKAYERNELAPNKPDEREYERRLRESYEGGYVNEPEKGLWEGIVSLDFRSSGSSIIITHNVSPDTLNRENCKEYDVAPQVGHRFCKDFPGFIPSLLGNLLEERQKIKKRMKESKDPVEKKLLDYRQRLIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLRYQKTKQVGLGAWLKF

SEQ ID NO: 3 - protein, 774 aa, Pyrococcus abyssi
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVAIATLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPAIRRTINLPTYTLETVYEVIFGKSKEKVYAHEIAEAWETGKGLERVAKYSMEDAKVTSELGKEFFPMEAQLARLVGHPVWDVSRSSTGNLVEWFLLTKAYERNELAPNKPDEREYERRLRESYEGGYVNEPEKGLWEGIVSLDFRSQAISIIITHNVSPDTLNRENCKEYDVAPQVGHRFCKDFPGFIPSLLGNLLEERQKIKKRMKESKDPVEKKLLDYRQRLIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLRYQKTKQVGLGAWLKFSGS

SEQ ID NO: 4 - protein, 774 aa, Pyrococcus abyssi
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVAIATLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPAIRRTINLPTYTLETVYEVIFGKSKEKVYAHEIAEAWETGKGLERVAKYSMEDAKVTSELGKEFFPMEAQLARLVGHPVWDVSRSSTGNLVEWFLLTKAYERNELAPNKPDEREYERRLRESYEGGYVNEPEKGLWEGIVSLDFRSYSCSIIITHNVSPDTLNRENCKEYDVAPQVGHRFCKDFPGFIPSLLGNLLEERQKIKKRMKESKDPVEKKLLDYRQRLIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLRYQKTKQVGLGAWLKFSGS

SEQ ID NO: 5 - protein, 774 aa, Pyrococcus abyssi
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVAIATLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPAIRRTINLPTYTLETVYEVIFGKSKEKVYAHEIAEAWETGKGLERVAKYSMEDAKVTSELGKEFFPMEAQLARLVGHPVWDVSRSSTGNLVEWFLLTKAYERNELAPNKPDEREYERRLRESYEGGYVNEPEKGLWEGIVSLDFRSFSASIIITHNVSPDTLNRENCKEYDVAPQVGHRFCKDFPGFIPSLLGNLLEERQKIKKRMKESKDPVEKKLLDYRQRLIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLRYQKTKQVGLGAWLKFSGS

(19) United States
(12) Patent Application Publication
(10) Pub. No.: US 2022/0145272 A1
(43) Pub. Date: May 12, 2022

(54) POLYMERASE ENZYME FROM PYROCOCCUS ABYSSI

(71) Applicant: IsoPlexis Corporation, Brandford, CT (US)

(72) Inventors: Jerzy Olejnik, Brookline, MA (US); Angela Delucia, Cambridge, MA (US); Cheng-yao Chen, Eugene, OR (US); Ryan Charles Heller, Amesbury, MA (US); Thomas William Schoenfeld, Topsfield, MA (US)

(21) Appl. No.: 16/485,282

(22) PCT Filed: Feb. 13, 2018
(86) PCT No.: PCT/US2018/017993

§ 371 Date: Aug. 12, 2019

Related U.S. Application Data
(60) Provisional application No. 62/458,385, filed on Feb. 13, 2017.

(30) Foreign Application Priority Data
Mar. 10, 2017 (EP) 17160391.3

(51) Int. Cl. C12N 9/12

(52) U.S. Cl. CPC C12N 9/1252; C12Y 207/07007

(57) ABSTRACT

The present invention relates to a polymerase enzyme with improved ability to incorporate reversibly terminating nucleotides. The enzyme comprising the following mutations in the motif A region (SGS). It relates to a polymerase enzyme according to SEQ ID NO. 1 with mutations in the amino acid sequence positions 409, 410 and 411.

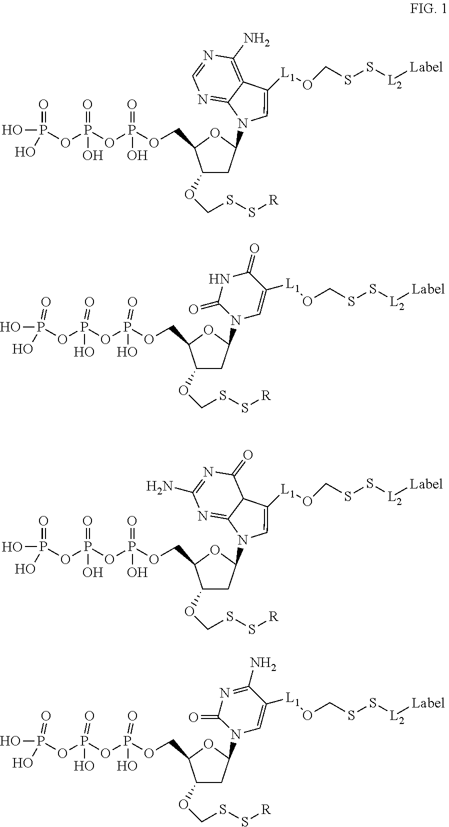

### FIELD OF THE INVENTION

The present invention is in the field of molecular biology, in particular in the field of enzymes and more particular in the field of polymerases. It is also in the field of nucleic acid sequencing.

### BACKGROUND

The invention relates to polymerase enzymes, in particular modified DNA polymerases which show improved incorporation of modified nucleotides compared to a control polymerase. Also included in the present invention are methods of using the modified polymerases for DNA sequencing, in particular next generation sequencing.

Three main super families of DNA polymerase exist, based upon their amino acid similarity to E. coli DNA polymerases I, II and III. They are called family A, B and C polymerases respectively. Whilst crystallographic analysis of Family A and B polymerases reveals a common structural core for the nucleotide binding site, sequence motifs that are well conserved within families are only weakly conserved between families, and there are significant differences in the way these polymerases discriminate between nucleotide analogues. Early experiments with DNA polymerases revealed difficulties incorporating modified nucleotides such as dideoxynucleotides (ddNTPs). There are, therefore, several examples in which DNA polymerases have been modified to increase the rates of incorporation of nucleotide analogues. The majority of these have focused on variants of Family A polymerases with the aim of increasing the incorporation of dideoxynucleotide chain terminators. For example, Tabor, S. and Richardson, C. C. ((1995) Proc. Natl. Acad. Sci (USA) 92:6339) describe the replacement of phenylalanine 667 with tyrosine in T. aquaticus DNA polymerase and the effects this has on discrimination of dideoxynucleotides by the DNA polymerase.

In order to increase the efficiency of incorporation of modified nucleotides, DNA polymerases have been utilised or engineered such that they lack 3′-5′ exonuclease activity (designated exo-). The exo-variant of 9° N polymerase is described by Perler et al., 1998 U.S. Pat. No. 5,756,334 and by Southworth et al., 1996 Proc. Natl Acad. Sci USA 93:5281.

Gardner A. F. and Jack W. E. (Determinants of nucleotide sugar recognition in an archaeon DNA polymerase Nucl. Acids Res. 27:2545, 1999) describe mutations in Vent DNA polymerase that enhance the incorporation of ribo-, 2′ and 3′deoxyribo- and 2′-3′-dideoxy-ribonucleotides. The two individual mutations in Vent polymerase, Y412V and A488L, enhanced the relative activity of the enzyme with the nucleotide ATP. In addition, other substitutions at Y412 and A488 also increased ribonucleotide incorporation, though to a lesser degree. It was concluded that the bulk of the amino acid side chain at residue 412 acts as a “steric gate” to block access of the 2′-hydroxyl of the ribonucleotide sugar to the binding site. However, the rate enhancement with cordycepin (3′deoxy adenosine triphosphate) was only 2-fold, suggesting that the Y412V polymerase variant was also sensitive to the loss of the 3′ sugar hydroxyl. For residue A488, the change in activity is less easily rationalized. A488 is predicted to point away from the nucleotide binding site; here the enhancement in activity was explained through a change to the activation energy required for the enzymatic reaction. These mutations in Vent correspond to Y409 and A485 in 9° N polymerase.

The universality of the A488L mutation in conferring reduced discrimination against nucleotide analogs has been confirmed by homologous mutations in the following hyperthermophilic polymerases:

A486Y variant of Pfu DNA polymerase (Evans et al., 2000. Nucl. Acids. Res. 28:1059). A series of random mutations was introduced into the polymerase gene and variants were identified that had improved incorporation of ddNTPs. The A486Y mutation improved the ratio of ddNTP/dNTP in sequencing ladders by 150-fold compared to wild type. However, mutation of Y410 to A or F produced a variant that resulted in an inferior sequencing ladder compared to the wild type enzyme. For further information, reference is made to International Publication No. WO 01/38546.

A485L variant of 9° N DNA polymerase (Gardner and Jack, 2002. Nucl. Acids Res. 30:605). This study demonstrated that the mutation of Alanine to Leucine at amino acid 485 enhanced the incorporation of nucleotide analogues that lack a 3′ sugar hydroxyl moiety (acyNTPs and dideoxyNTPs).

A485T variant of Tsp JDF-3 DNA polymerase (Arezi et al., 2002. J. Mol. Biol. 322:719). In this paper, random mutations were introduced into the JDF-3 polymerase from which variants were identified that had enhanced incorporation of ddNTPs. Individually, two mutations, A485T and P410L, improved ddNTP uptake compared to the wild type enzyme. In combination, these mutations had an additive effect and improved ddNTP incorporation by 250-fold. This paper demonstrates that the simultaneous mutation of two regions of a DNA polymerase can have additive effects on nucleotide analogue incorporation. In addition, this report demonstrates that P410, which lies adjacent to Y409 described above, also plays a role in the discrimination of nucleotide sugar analogues.

WO 01/23411 describes the use of the A488L variant of Vent in the incorporation of dideoxynucleotides and acyclonucleotides into DNA. The application also covers methods of sequencing that employ these nucleotide analogues and variants of 9° N DNA polymerase that are mutated at residue 485.

WO 2005/024010 A1 also relates to the modification of the motif A region and to the 9° N DNA polymerase. EP 1 664 287 B1 also relates to various altered family B type archeal polymerase enzymes which is capable of improved incorporation of nucleotides which have been modified at the 3′ sugar hydroxyl such that the substituent is larger in size than the naturally occurring 3′ hydroxyl group, compared to a control family B type archeal polymerase enzyme.

Yet, the modifications today still do not show sufficiently high incorporation rates of modified nucleotides (3′OH substituted analogs or having both substitutions on 3′-OH and carrying labels at the base). It would therefore be beneficial in order to improve sequencing performance to have enzymes that have such high incorporation rates of variety of modified nucleotides. One additional feature that is desirable is the tolerance for base modifications. For example, labels can be attached to the base or the 3′-OH via cleavable or non-cleavable linkers. In case of cleavable linkers attached to the base, there is usually a residual spacer arm left after the cleavage. This residual modification may interfere with incorporation of subsequent nucleotides by polymerase. Therefore, it is highly desirable to have polymerases for carrying out sequencing by synthesis process (SBS) that are tolerable of these scars.

### SUMMARY OF THE INVENTION

To improve the efficiency of certain DNA sequencing methods, the inventors have attempted to look for organisms other than 9° N. Also, to improve the efficiency of certain DNA sequencing methods, the inventors have analyzed whether such other DNA polymerases could be modified to produce improved rates of incorporation of such 3′ substituted nucleotide analogues.

The invention relates to a Polymerase enzyme according to SEQ ID NO. 1 or any polymerase that shares at least 70%, 80%, 90%, 95% or, 98% amino acid sequence identity thereto, comprising the following mutation(s):
a. at position 409 of SEQ ID NO. 1:
   i. serine (S) (L409S) or,
   ii. glutamine (Q) (L409Q) or,
   iii. tyrosine (Y) (L409Y) or,
   iv. phenylalanine (F) (L409F)
b. at position 410 of SEQ ID NO. 1:
   i. glycine (G) (Y410G) or,
   ii. alanine (A) (Y409A) or,
   iii. serine (S) (Y409S),
c. at position 411 of SEQ ID NO. 1:
   i. serine (S) (P411S) or,
   ii. isoleucine (I) (P411I) or,
   iii. cysteine (C) (P411C) or,
   iv. alanine (A) (P411A),
wherein the enzyme has little or no 3′-5′ exonuclease activity.

The invention relates to a polymerase enzyme according to SEQ ID NO. 1 or any polymerase that shares at least 70%, 80%, 90%, 95%, 98% amino acid sequence identity thereto, comprising a mutation selected from the group of: (i) at position 409 of SEQ ID NO. 1: glutamine (Q) (L409S), (ii) at position 410 of SEQ ID NO. 1: glycine (G) and/or (Y410G), (iii) at position 411 of SEQ ID NO. 1: serine (S) (P411S), wherein the enzyme has little or no 3′-5′ exonuclease activity. In one embodiment polymerases also carry modifications/substitutions at position 486. Particularly preferred substitution is A->L. Substitutions at this position exhibit synergy with substitutions at positions 409/410/411 and additional substitution at position 486. Particularly preferred substitution is A->L (A486L). Substitutions at this position exhibit synergy with substitutions at positions 409/410/411 in P. abyssi derived polymerase.

The invention relates to a polymerase enzyme according to SEQ ID NO. 1 or any polymerase that shares at least 70%, 80%, 90%, 95%, 98% amino acid sequence identity thereto, comprising a mutation set selected from the group of:

| 409 | 410 | 411 |
|---|---|---|
| S | G | S |
| Q | A | I |
| Y | S | C |
| F | S | A |

The invention also relates to the use of a modified polymerase in DNA sequencing and a kit comprising such an enzyme.

Herein, “incorporation” means joining of the modified nucleotide to the free 3′ hydroxyl group of a second nucleotide via formation of a phosphodiester linkage with the 5′ phosphate group of the modified nucleotide. The second nucleotide to which the modified nucleotide is joined will typically occur at the 3′ end of a polynucleotide chain.

Herein, “modified nucleotides” and “nucleotide analogues” when used in the context of this invention refer to nucleotides which have been modified at the 3′ sugar hydroxyl such that the substituent is larger in size than the naturally occurring 3′ hydroxyl group. In addition, these nucleotides may carry additional modifications, such as detectable labels attached to the base moiety. These terms may be used interchangeably.

Herein, the term “large 3′ substituent(s)” refers to a substituent group at the 3′ sugar hydroxyl which is larger in size than the naturally occurring 3′ hydroxyl group.

Herein, “improved” incorporation is defined to include an increase in the efficiency and/or observed rate of incorporation of at least one modified nucleotide, compared to a control polymerase enzyme. However, the invention is not limited just to improvements in absolute rate of incorporation of the modified nucleotides. As shown below the polymerases also incorporate other modifications and so called dark nucleotides nucleotides (non-labeled, terminating or reversibly terminating), hence, “improved incorporation” is to be interpreted accordingly as also encompassing improvements in any of these other properties, with or without an increase in the rate of incorporation. For example, tolerance for modifications on the bases could be the result of the improved properties as could be ability to incorporate modified nucleotides at a range of concentrations and temperatures. The “improvement” need not be constant over all cycles. Herein, “improvement” may be the ability to incorporate the modified nucleotides at low temperatures and/or over a wider temperature range than the control enzyme. Herein, “improvement” may be the ability to incorporate the modified nucleotides when using a lower concentration of the modified nucleotides as substrate or lower concentration of polymerase. Preferably the altered polymerase should exhibit detectable incorporation of the modified nucleotide when working at a substrate concentration in the nanomolar range.

Herein, “altered polymerase enzyme” means that the polymerase has at least one amino acid change compared to the control polymerase enzyme. In general, this change will comprise the substitution of at least one amino acid for another. In certain instances, these changes will be conservative changes, to maintain the overall charge distribution of the protein. However, the invention is not limited to only conservative substitutions. Non-conservative substitutions are also envisaged in the present invention. Moreover, it is within the contemplation of the present invention that the modification in the polymerase sequence may be a deletion or addition of one or more amino acids from or to the protein, provided that the polymerase has improved activity (over e.g. the wildtype) with respect to the incorporation of nucleotides modified at the 3′ sugar hydroxyl such that the substituent is larger in size than the naturally occurring 3′ hydroxyl group as compared to a control polymerase enzyme, such as the wild type according to SEQ ID NO. 1 lacking the 3′-5′ exonuclease activity.

The control polymerase may comprise any one of the listed substitution mutations functionally equivalent to the amino acid sequence of the given base polymerase (or an exo-variant thereof). Thus, the control polymerase may be a mutant version of the listed base polymerase having one of the stated mutations or combinations of mutations, and preferably having amino acid sequence identical to that of the base polymerase (or an exo-variant thereof) other than at the mutations recited above. Alternatively, the control polymerase may be a homologous mutant version of a polymerase other than the stated base polymerase, which includes a functionally equivalent or homologous mutation (or combination of mutations) to those recited in relation to the amino acid sequence of the base polymerase. By way of illustration, the control polymerase could be a mutant version of the Pfu polymerase having one of the mutations or combinations of mutations listed as optional or preferable above and below relative to the Pfu amino acid sequence or a P. abyssi polymerase or a mutant thereof or it could be a mutant version of another polymerase. It would however not comprise the S-G-S mutation claimed herein.

Alternatively the control polymerase is the P. abyssi wildtype polymerase with the SEQ ID No: 1.

As used herein, the term, “nucleotide” comprises a purine or pyrimidine base linked glycosidically to a sugar (ribose or deoxyribose), and one or more phosphate groups attached to the 5′ position of the sugar. “Nucleosides”, as used herein, comprise a purine or pyrimidine base linked glycosidically to a sugar (ribose or deoxyribose), but lack a phosphate group at the 5′ position of the sugar. With respect to the method claims described herein, it is generally understood that a nucleoside (lacking a 5′ phosphate group) cannot be incorporated by a polymerase. Synthetic and naturally occurring nucleotides, prior to their modification at the 3′ sugar hydroxyl, are included within the definition. Labeling of the bases can occur via naturally occurring groups (such as exocyclic amines for adenosine or guanosine) or via modifications, such as 5- and 7-deaza analogs. One preferred embodiment is attachment via 5-(pyrimidines) and 7-deaza (purines) propynyl group, more preferably propargylamine or propargylhydroxy group. Another preferred attachment is via hydroxymethyl groups as disclosed in U.S. Pat. No. 9,322,050.

Herein, and throughout the specification mutations within the amino acid sequence of a polymerase are written in the following form: (i) single letter amino acid as found in wild type polymerase, (ii) position of the change in the amino acid sequence of the polymerase and (iii) single letter amino acid as found in the altered polymerase. So, mutation of a Tyrosine residue in the wild type polymerase to a Valine residue in the altered polymerase at position 409 of the amino acid sequence would be written as Y409V. This is standard procedure in molecular biology.

### DETAILED DESCRIPTION OF THE INVENTION

The sheer increase in rates of incorporation of the modified analogues that have been achieved with polymerases of the invention is unexpected. The examples show that even existing polymerases with mutations do not exhibit these high incorporation rates.

The invention relates to a polymerase enzyme according to SEQ ID NO. 1 or any polymerase that shares at least 70% amino acid sequence identity thereto, comprising a mutation selected from the group of: (i) at position 409 of SEQ ID NO. 1: serine (S) and/or (L409S), (ii) at position 410 of SEQ ID NO. 1: glycine (G) and/or (Y410G), (iii) at position 411 of SEQ ID NO. 1: serine (S) (P411S), wherein the enzyme has little or no 3′-5′ exonuclease activity.

Preferably, the enzyme claimed shares 75%, 80%, 85%, 90%, 95%, 98%, 99%, 99.5% or 100% sequence identity with the enzyme according to SEQ ID NO. 1. These percentages do not include the additionally claimed mutations.

The altered polymerase will generally and preferably be an “isolated” or “purified” polypeptide. By “isolated polypeptide” a polypeptide that is essentially free from contaminating cellular components is meant, such as carbohydrates, lipids, nucleic acids or other proteinaceous impurities which may be associated with the polypeptide in nature. One may use a His-tag for purification, but other means may also be used. Preferably, at least the altered polymerase may be a “recombinant” polypeptide.

The altered polymerase according to the invention may be a family B type DNA polymerase, or a mutant or variant thereof. Family B DNA polymerases include numerous archaeal DNA polymerase, human DNA polymerase a and T4, RB69 and φ29 phage DNA polymerases. Family A polymerases include polymerases such as Taq, and T7 DNA polymerase. In one embodiment the polymerase is selected from any family B archaeal DNA polymerase, human DNA polymerase a or T4, RB69 and φ29 phage DNA polymerases.

Preferably, the polymerase is from an organism belonging to the family of Thermococcaceae, preferably from the genera of Pyrococcus. Such organisms include, Pyrococcus abyssi, Pyrococcus woesei, Pyrococcus yayanosii, Pyrococcus horikoshii, Pyrococcus furiosus or, e.g. Pryococcus glycovorans, Pyrococcus glycovorans, Thermococcus zilligii, Thermococcus sp. 4557. Most preferably the enzyme is from Pyrococcus abyssi.

Ideally, the polymerase comprises all of the following mutations, L409S, Y410G and P411S and optionally additionally, comprises one or more of the following additional mutations or equivalent mutations in other polymerase families: D141A, E143A, A486L. Mutations at 141/143 positions are known to eliminate most of the exonuclease proofreading ability. Mutations at position 486 are known to enhance incorporation of non-native nucleotides (terminator mutations); see Gardner and Jack, 2002. Nucl. Acids Res. 30:605.

Preferably, the enzyme additionally comprises a mutation A486L

Preferred is a polymerase, wherein the enzyme shares 95%, preferably even 98% sequence identity (not counting the mutations) with SEQ ID NO. 1 and additionally has the following set of mutations, (i) L409S, Y410G, P411S and (ii) A486L.

In a preferred embodiment the enzyme is selected from the group of SEQ ID NO. 2 to 5. Preferred is a polymerase, wherein the enzyme shares 95%, preferably even 98% sequence identity with SEQ ID NO. 2 to 5. A very preferred enzyme is that with SEQ ID NO. 2.

Preferably, the modified polymerase comprises a mutation corresponding to A485L in 9° N polymerase. This mutation corresponds to A488L in Vent and A486L in Pfu and P. abyssi. Several other groups have published on this mutation. A486Y variant of Pfu DNA polymerase (Evans et al., 2000. Nucl. Acids. Res. 28:1059). A series of random mutations was introduced into the polymerase gene and variants were identified that had improved incorporation of ddNTPs. The A486Y mutation improved the ratio of ddNTP/dNTP in sequencing ladders by 150-fold compared to wild type. However, mutation of Y410 to A or F produced a variant that resulted in an inferior sequencing ladder compared to the wild type enzyme; see also WO 01/38546. A485L variant of 9° N DNA polymerase (Gardner and Jack, 2002. Nucl. Acids Res. 30:605). This study demonstrated that the mutation of Alanine to Leucine at amino acid 485 enhanced the incorporation of nucleotide analogues that lack a 3′ sugar hydroxyl moiety (acyNTPs and dideoxyNTPs). A485T variant of Tsp JDF-3 DNA polymerase (Arezi et al., 2002. J. Mol. Biol. 322:719). In this paper, random mutations were introduced into the JDF-3 polymerase from which variants were identified that had enhanced incorporation of ddNTPs. WO 01/23411 describes the use of the A488L variant of Vent in the incorporation of dideoxynucleotides and acyclonucleotides into DNA. The application also covers methods of sequencing that employ these nucleotide analogues and variants of 9° N DNA polymerase that are mutated at residue 485.

The invention relates to a polymerase with the mutations shown herein which exhibits an increased rate of incorporation of nucleotides which have been modified at the 3′ sugar hydroxyl such that the substituent is larger in size than the naturally occurring 3′ hydroxyl group and ddNTPs, compared to the control polymerase being a normal unmodified enzyme.

Such nucleotides are disclosed in WO 2004/018497 A2. Here, a modified nucleotide molecule comprising a purine or pyrimidine base and a ribose or deoxyribose sugar moiety having a removable 3′-OH blocking group covalently attached thereto, such that the 3′ carbon atom has attached a group of the structure: —O—Z is disclosed, wherein Z is any of —C(R′)₂—N(R″)₂′C(R′)₂—N(H)R″, and —C(R′)₂—N₃, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; or (R′)₂ represents an alkylidene group of formula ═C(R′″)₂ wherein each R′″ may be the same or different and is selected from the group comprising hydrogen and halogen atoms and alkyl groups; and wherein said molecule may be reacted to yield an intermediate in which each R″ is exchanged for H, which intermediate dissociates under aqueous conditions to afford a molecule with a free 3′OH.

The inventors have found that the claimed polymerase may be used in extension reactions and sequencing reactions very well when a novel nucleotide is used. Thus, the invention relates to a method of sequencing a nucleic acid wherein the claimed polymerase is used together with the following nucleotide.

In a preferred embodiment nucleotide has the following characteristics. The nucleotide comprises a nucleobase, a sugar, and at least one phosphate group at the 5′ position, wherein said nucleobase comprising a detectable label attached via a cleavable oxymethylenedisulfide linker, said sugar comprising a 3′-O capped by a cleavable protecting group comprising methylenedisulfide.

Ideally, the nucleobase is a non-natural nucleobase and is selected from the group comprising 7-deaza guanine, 7-deaza adenine, 2-amino, 7-deaza adenine, and 2-amino adenine.

Ideally, the cleavable protecting group is of the formula —CH2-SS—R, wherein R is selected from the group comprising alkyl and substituted alkyl groups.

Preferably, the nucleotide has this structure:

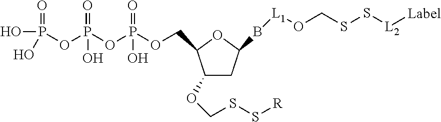

Here, B is a nucleobase, R is selected from the group comprising alkyl and substituted alkyl groups, and L1 and L2 are connecting groups. Preferably, L₁ and L₂ are independently selected from the group comprising —CO—, —CONH—, —NHCONH—, -0-, —S—, —ON, and —N═N—, alkyl, aryl, branched alkyl, branched aryl. Ideally L₁ and L₂ are the same.

The invention relates to a kit comprising a DNA polymerase as disclosed herein and claimed herein, and at least one nucleotide (e.g. a deoxynucleotide triphosphate) comprising a nucleobase, a sugar, and at least one phosphate group at the 5′ position, wherein said sugar comprising a cleavable protecting group on the 3′-O, wherein said cleavable protecting group comprises methylenedisulfide, and wherein said nucleotide further comprises a detectable label attached via a cleavable oxymethylenedisulfide linker to the nucleobase of said nucleotide.

Claimed is also a reaction mixture comprising a nucleic acid template with a primer hybridized to said template, a DNA polymerase according to the invention and at least one nucleotide comprising a nucleobase, a sugar, and at least one phosphate group at the 5′ position, wherein said sugar comprising a cleavable protecting group on the 3′-O, wherein said cleavable protecting group comprises methylenedisulfide, wherein said nucleotide further comprises a detectable label attached via a cleavable oxymethylenedisulfide linker to the nucleobase of said nucleotide.

Claimed is a method of performing a DNA synthesis reaction comprising the steps of a) providing a nucleic acid template with a primer hybridized to said template, the DNA polymerase according to the invention, at least one nucleotide comprising a nucleobase a sugar, and at least one phosphate group at the 5′ position, wherein said sugar comprising a cleavable protecting group on the 3′-O, wherein said cleavable protecting group comprises methylenedisulfide, wherein said nucleotide further comprises a detectable label attached via a cleavable oxymethylenedisulfide linker to the nucleobase of said nucleotide, and b) subjecting said reaction mixture to conditions which enable a DNA polymerase catalyzed primer extension reaction.

The invention also relates to a method for analyzing a DNA sequence comprising the steps of a) providing a nucleic acid template with a primer hybridized to said template forming a primer/template hybridization complex, b) adding DNA polymerase according to the invention, and a first nucleotide comprising a nucleobase, a sugar, and at least one phosphate group at the 5′ position, wherein said sugar comprising a cleavable protecting group on the 3′-O, wherein said cleavable protecting group comprises methylenedisulfide, wherein said nucleotide further comprises a first detectable label attached via a cleavable oxymethylenedisulfide linker to the nucleobase of said nucleotide, c) subjecting said reaction mixture to conditions which enable a DNA polymerase catalyzed primer extension reaction so as to create a modified primer/template hybridization complex, and d) detecting a said first detectable label of said nucleotide in said modified primer/template hybridization complex. The blocking group may be repeatedly removed and novel nucleotides added. These methods are known to the person skilled in the art. Here, differently labeled, 3′-O methylenedisulfide capped nucleotide compounds representing analogs of A, G, C and T or U are used in step b). Ideally, step e) is performed by exposing said modified primer/template hybridization complex to a reducing agent. A variety of reducing agents can be used that are capable of cleaving the disulfide bonds. For example, thiol compounds (cysteine, cysteamine), dithiol compounds (dithiothreitol, dimercaptopropane sulfate), phosphines (TCEP, tris-hysdroxypropyl phosphine) can be used for this purpose.

In another embodiment the labelled nucleotide that is used is as follows.

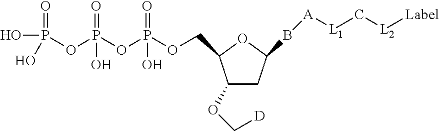

Here, D is selected from the group consisting of an azide, disulfide alkyl and disulfide substituted alkyl groups, B is a nucleobase, A is an attachment group, C is a cleavable site core, L₁ and L₂ are connecting groups, and Label is a label. Ideally, the nucleobase is selected from the group of 7-deaza guanine, 7-deaza adenine, 2-amino, 7-deaza adenine, and 2-amino adenine.

L₁ is selected from the group consisting of —CONH(CH₂)ₓ— —CO—O(CH₂)ₓ— —CONH—(OCH₂CH₂0)ₓ-CO—O(CH₂CH₂0)ₓ- and —CO(CH₂)ₓ— wherein x is 0-10. L₂ can be,

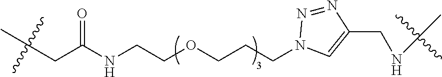

L₂ can be, —NH—, —(CH₂)_{X}—NH—, —C(Me)₂(CH₂)ₓNH—, —CH(Me)(CH₂)ₓNH—, —C(Me)₂(CH₂)ₓCO, —CH(Me)(CH₂)ₓCO—, —(CH₂)ₓOCONH(CH₂)_{y}O(CH₂)_{z}NH—, —(CH₂)ₓCONH(CH₂CH₂O)_{y}(CH₂)_{z}NH—, and —CONH(CH₂)ₓ—, —CO(CH₂)ₓ— wherein x, y, and z are each independently selected from is 0-10.

Preferably the labelled nucleotide has the following structure:

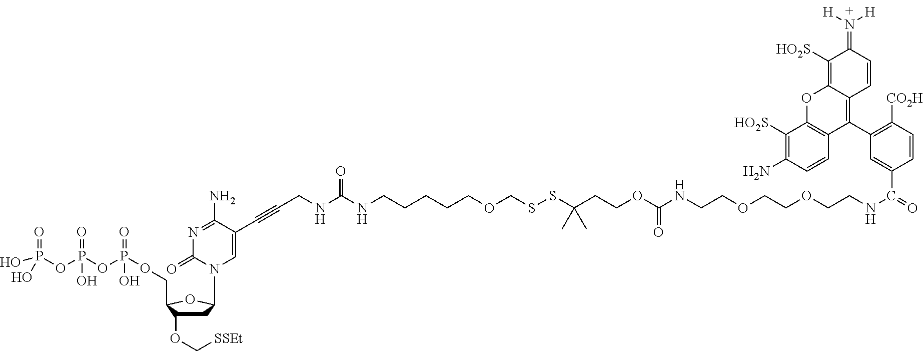

Preferably the labelled nucleotide has the following structure:

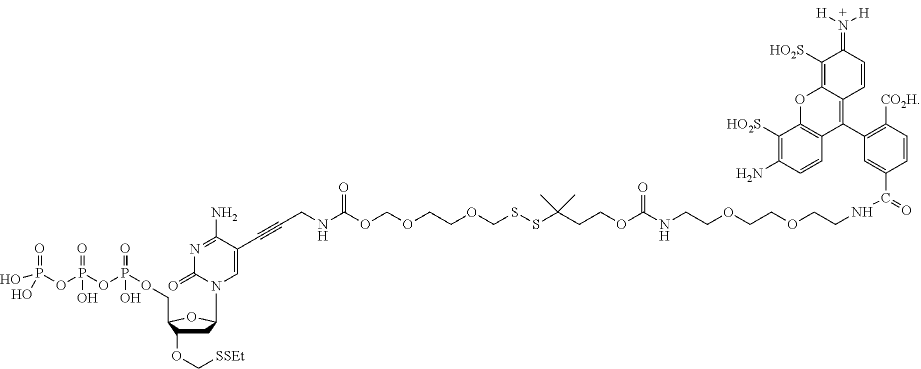

Preferably the labelled nucleotide has the following structure:

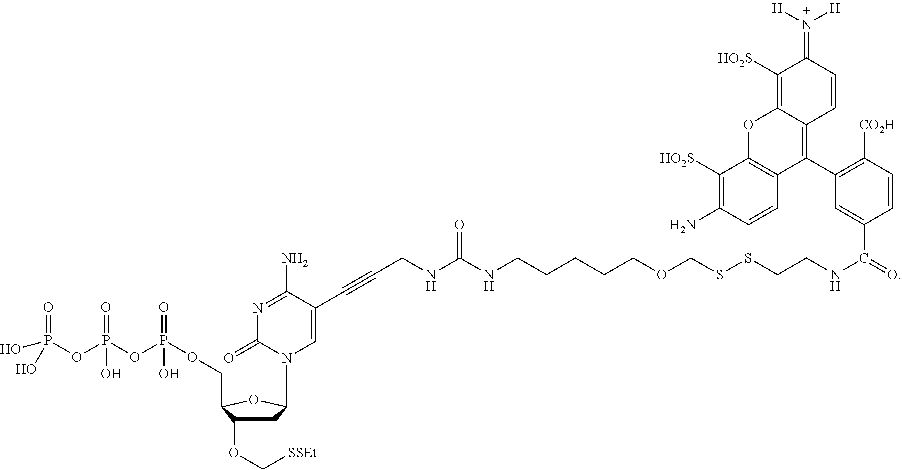

Preferably the labelled nucleotide has the following structure:

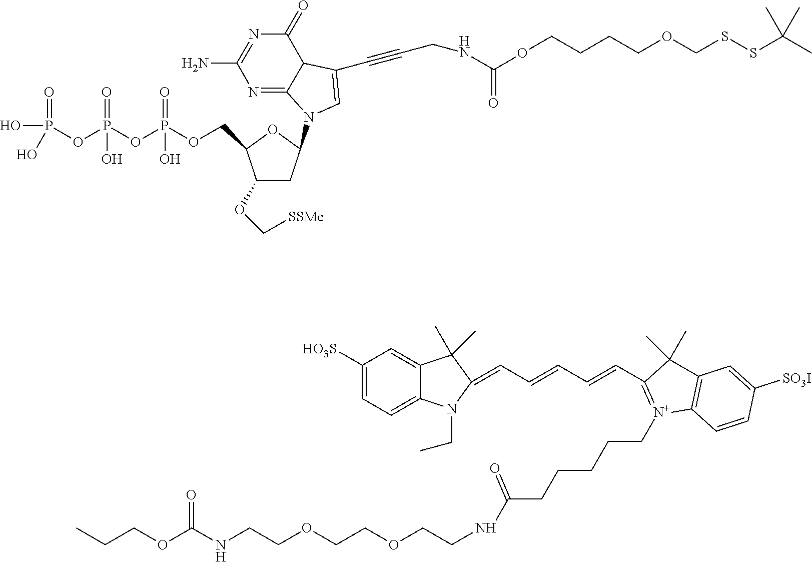

Preferably the labelled nucleotide has the following structure:

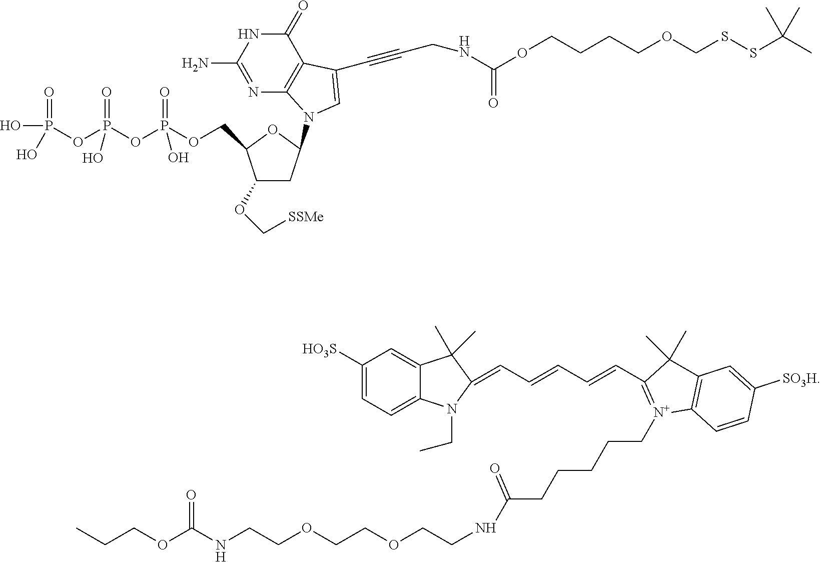

Preferably the labelled nucleotide has the following structure:

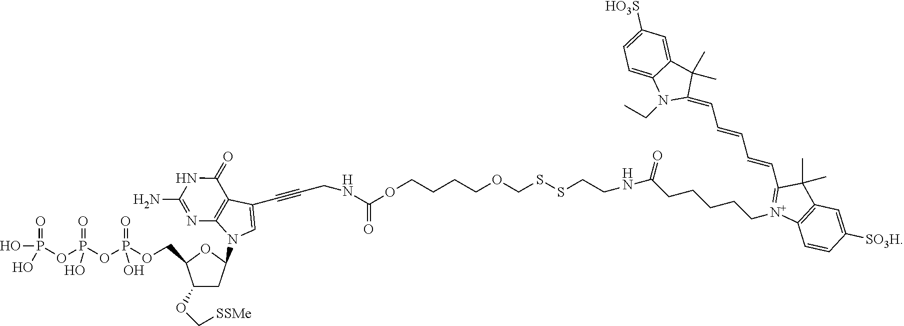

Preferably the labelled nucleotide has the following structure:

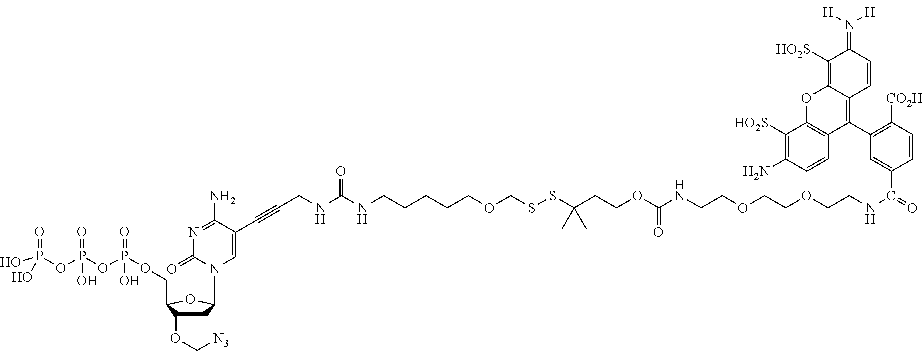

Preferably the labelled nucleotide has the following structure:

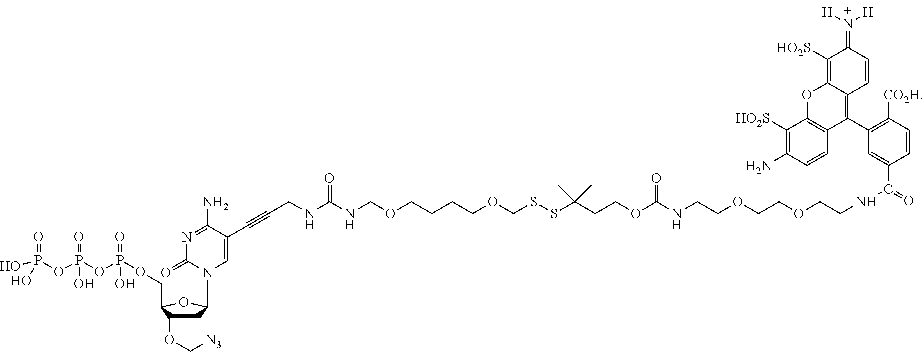

Preferably the labelled nucleotide has the following structure:

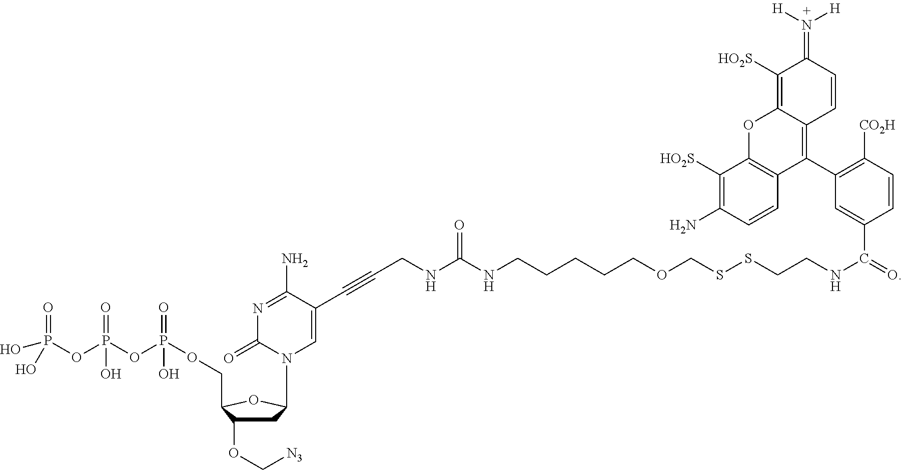

Preferably the labelled nucleotides have the following structures:

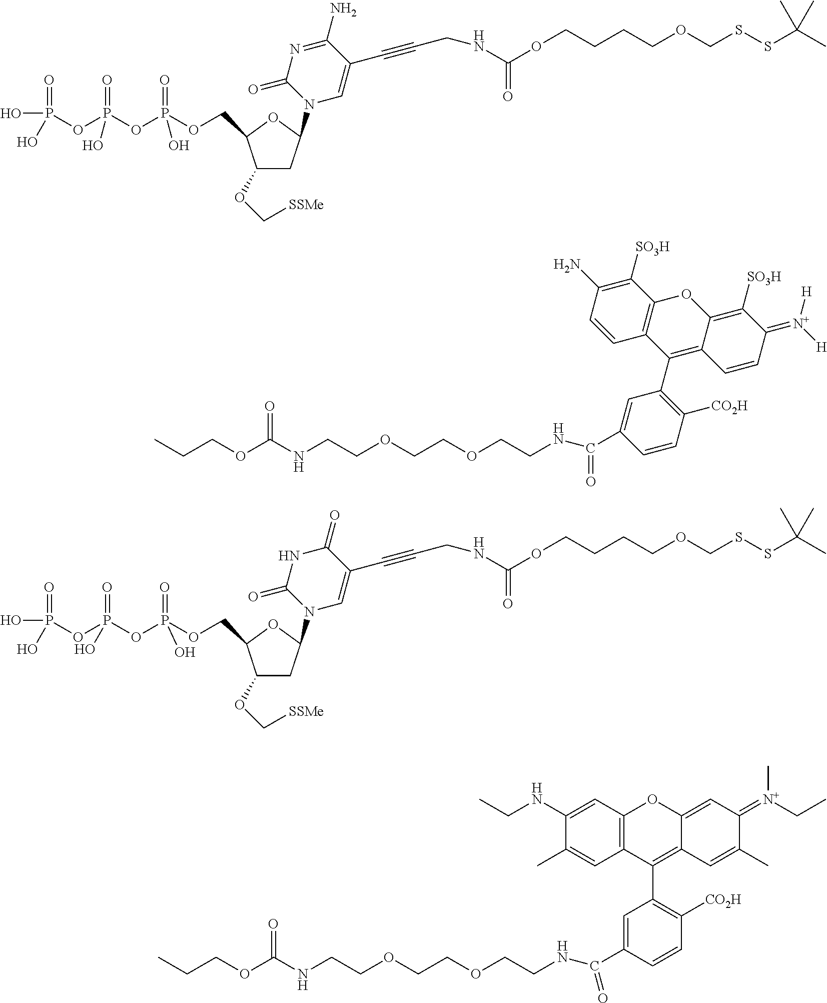

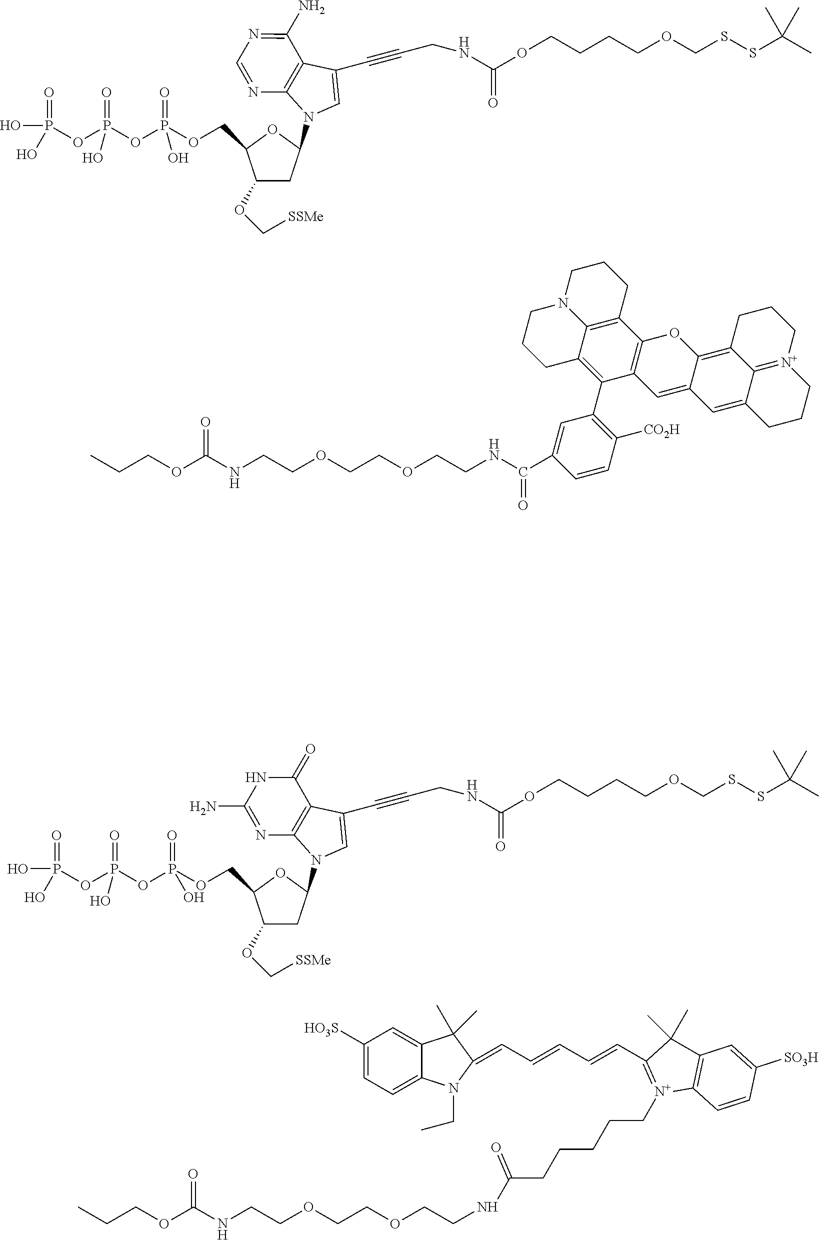

Preferably the non labelled nucleotides have the following structures:

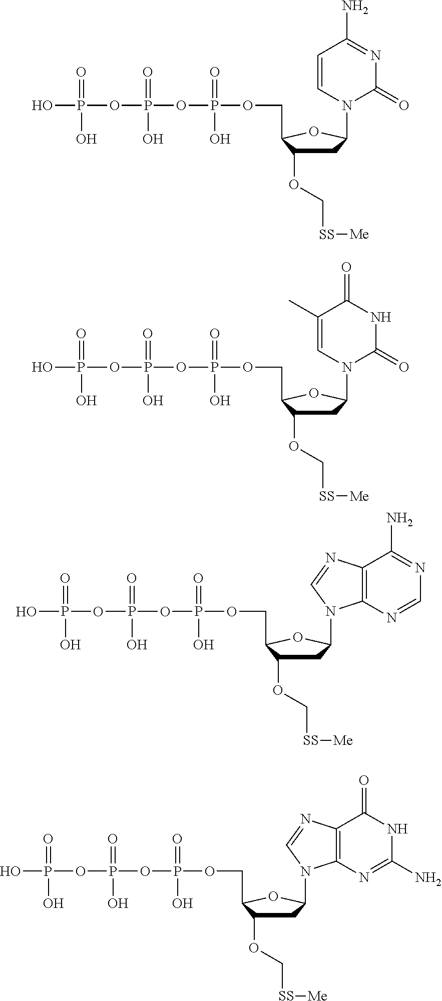

The invention also relates to a nucleic acid molecule encoding a polymerase according to the invention, as well as an expression vector comprising said nucleic acid molecule.

The invention also relates to a method for incorporating nucleotides which have been modified at the 3′ sugar hydroxyl such that the substituent is larger in size than the naturally occurring 3′ hydroxyl group into DNA comprising the following substances (i) a polymerase according to any one of the previous embodiments, (ii) template DNA, (iii) one or more nucleotides, which have been modified at the 3′ sugar hydroxyl such that the substituent is larger in size than the naturally occurring 3′ hydroxyl group.

The invention also relates to a method for incorporating nucleotides which have been modified at the 3′ sugar hydroxyl such that the substituent is larger in size than the naturally occurring 3′ hydroxyl group into DNA comprising the following substances (i) a polymerase according to any one of the previous embodiments, (ii) template DNA, (iii) one or more nucleotides, which have been modified at the 3′ sugar hydroxyl such that the substituent is larger in size than the naturally occurring 3′ hydroxyl group, wherein the blocking group comprises a disulfide preferably, methylenedisulfide.

The invention also relates to the use of a polymerase according to the invention in methods such as nucleic acid labeling, or sequencing. The polymerases of the present invention are useful in a variety of techniques requiring incorporation of a nucleotide into a polynucleotide, which include sequencing reactions, polynucleotide synthesis, nucleic acid amplification, nucleic acid hybridization assays, single nucleotide polymorphism studies, and other such techniques. All such uses and methods utilizing the modified polymerases of the invention are included within the scope of the present invention.

In sequencing the use of nucleotides bearing a 3′ block allows successive nucleotides to be incorporated into a polynucleotide chain in a controlled manner. After each nucleotide addition the presence of the 3′ block prevents incorporation of a further nucleotide into the chain. Once the nature of the incorporated nucleotide has been determined, the block may be removed, leaving a free 3′ hydroxyl group for addition of the next nucleotide. Sequencing by synthesis of DNA ideally requires the controlled (i.e. one at a time) incorporation of the correct complementary nucleotide opposite the oligonucleotide being sequenced. This allows for accurate sequencing by adding nucleotides in multiple cycles as each nucleotide residue is sequenced one at a time, thus preventing an uncontrolled series of incorporations occurring. The incorporated nucleotide is read using an appropriate label attached thereto before removal of the label moiety and the subsequent next round of sequencing. In order to ensure only a single incorporation occurs, a structural modification (“blocking group”) of the sequencing nucleotides is required to ensure a single nucleotide incorporation but which then prevents any further nucleotide incorporation into the polynucleotide chain. The blocking group must then be removable, under reaction conditions which do not interfere with the integrity of the DNA being sequenced. The sequencing cycle can then continue with the incorporation of the next blocked, labelled nucleotide. In order to be of practical use, the entire process should consist of high yielding, highly specific chemical and enzymatic steps to facilitate multiple cycles of sequencing. To be useful in DNA sequencing, a nucleotide, and more usually nucleotide triphosphates, generally require a 3 OH-blocking group so as to prevent the polymerase used to incorporate it into a polynucleotide chain from continuing to replicate once the base on the nucleotide is added. The DNA template for a sequencing reaction will typically comprise a double-stranded region having a free 3′ hydroxyl group which serves as a primer or initiation point for the addition of further nucleotides in the sequencing reaction. The region of the DNA template to be sequenced will overhang this free 3′ hydroxyl group on the complementary strand. The primer bearing the free 3′ hydroxyl group may be added as a separate component (e.g. a short oligonucleotide) which hybridizes to a region of the template to be sequenced. Alternatively, the primer and the template strand to be sequenced may each form part of a partially self-complementary nucleic acid strand capable of forming an intramolecular duplex, such as for example a hairpin loop structure. Nucleotides are added successively to the free 3′ hydroxyl group, resulting in synthesis of a polynucleotide chain in the 5′ to 3′ direction. After each nucleotide addition the nature of the base which has been added will be determined, thus providing sequence information for the DNA template.

Such DNA sequencing may be possible if the modified nucleotides can act as chain terminators. Once the modified nucleotide has been incorporated into the growing polynucleotide chain complementary to the region of the template being sequenced there is no free 3′-OH group available to direct further sequence extension and therefore the polymerase can not add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

In a preferred embodiment the modified nucleotides carry a label to facilitate their detection. Preferably this is a fluorescent label. Each nucleotide type may carry a different fluorescent label. However, the detectable label need not be a fluorescent label. Any label can be used which allows the detection of the incorporation of the nucleotide into the DNA sequence.

One method for detecting the fluorescently labelled nucleotides, suitable for use in the second and third aspects of the invention, comprises using laser light of a wavelength specific for the labelled nucleotides, or the use of other suitable sources of illumination.

In one embodiment the fluorescence from the label on the nucleotide may be detected by a CCD camera.

If the DNA templates are immobilised on a surface they may preferably be immobilised on a surface to form a high density array. Most preferably, and in accordance with the technology developed by the applicants for the present invention, the high density array comprises a single molecule array, wherein there is a single DNA molecule at each discrete site that is detectable on the array. Single-molecule arrays comprised of nucleic acid molecules that are individually resolvable by optical means and the use of such arrays in sequencing are described, for example, in WO 00/06770, the contents of which are incorporated herein by reference. Single molecule arrays comprised of individually resolvable nucleic acid molecules including a hairpin loop structure are described in WO 01/57248, the contents of which are also incorporated herein by reference. The polymerases of the invention are suitable for use in conjunction with single molecule arrays prepared according to the disclosures of WO 00/06770 of WO 01/57248. However, it is to be understood that the scope of the invention is not intended to be limited to the use of the polymerases in connection with single molecule arrays. Single molecule array-based sequencing methods may work by adding fluorescently labelled modified nucleotides and an altered polymerase to the single molecule array. Complementary nucleotides would base-pair to the first base of each nucleotide fragment and would be added to the primer in a reaction catalysed by the improved polymerase enzyme. Remaining free nucleotides would be removed. Then, laser light of a specific wavelength for each modified nucleotide would excite the appropriate label on the incorporated modified nucleotides, leading to the fluorescence of the label. This fluorescence could be detected by a suitable CCD camera that can scan the entire array to identify the incorporated modified nucleotides on each fragment. Thus millions of sites could potentially be detected in parallel. Fluorescence could then be removed. The identity of the incorporated modified nucleotide would reveal the identity of the base in the sample sequence to which it is paired. The cycle of incorporation, detection and identification would then be repeated approximately 25 times to determine the first 25 bases in each oligonucleotide fragment attached to the array, which is detectable. Thus, by simultaneously sequencing all molecules on the array, which are detectable, the first 25 bases for the hundreds of millions of oligonucleotide fragments attached in single copy to the array could be determined. Obviously the invention is not limited to sequencing 25 bases. Many more or less bases could be sequenced depending on the level of detail of sequence information required and the complexity of the array. Using a suitable bioinformatics program the generated sequences could be aligned and compared to specific reference sequences. This would allow determination of any number of known and unknown genetic variations such as single nucleotide polymorphisms (SNPs) for example. The utility of the altered polymerases of the invention is not limited to sequencing applications using single-molecule arrays. The polymerases may be used in conjunction with any type of array-based (and particularly any high density array-based) sequencing technology requiring the use of a polymerase to incorporate nucleotides into a polynucleotide chain, and in particular any array-based sequencing technology which relies on the incorporation of modified nucleotides having large 3′ substituents (larger than natural hydroxyl group), such as 3′ blocking groups. The polymerases of the invention may be used for nucleic acid sequencing on essentially any type of array formed by immobilisation of nucleic acid molecules on a solid support. In addition to single molecule arrays suitable arrays may include, for example, multi-polynucleotide or clustered arrays in which distinct regions on the array comprise multiple copies of one individual polynucleotide molecule or even multiple copies of a small-number of different polynucleotide molecules (e.g. multiple copies of two complementary nucleic acid strands). In particular, the polymerases of the invention may be utilised in the nucleic acid sequencing method described in WO 98/44152, the contents of which are incorporated herein by reference. This International application describes a method of parallel sequencing of multiple templates located at distinct locations on a solid support. The method relies on incorporation of labelled nucleotides into a polynucleotide chain. The polymerases of the invention may be used in the method described in International Application WO 00/18957, the contents of which are incorporated herein by reference. This application describes a method of solid-phase nucleic acid amplification and sequencing in which a large number of distinct nucleic acid molecules are arrayed and amplified simultaneously at high density via formation of nucleic acid colonies and the nucleic acid colonies are subsequently sequenced. The altered polymerases of the invention may be utilised in the sequencing step of this method. Multi-polynucleotide or clustered arrays of nucleic acid molecules may be produced using techniques generally known in the art. By way of example, WO 98/44151 and WO 00/18957 both describe methods of nucleic acid amplification which allow amplification products to be immobilised on a solid support in order to form arrays comprised of clusters or “colonies” of immobilised nucleic acid molecules. The contents of WO 98/44151 and WO 00/18957 relating to the preparation of clustered arrays and use of such arrays as templates for nucleic acid sequencing are incorporated herein by reference. The nucleic acid molecules present on the clustered arrays prepared according to these methods are suitable templates for sequencing using the polymerases of the invention. However, the invention is not intended to use of the polymerases in sequencing reactions carried out on clustered arrays prepared according to these specific methods. The polymerases of the invention may further be used in methods of fluorescent in situ sequencing, such as that described by Mitra et al. Analytical Biochemistry 320, 55-65, 2003 and Lee et al, Nature Protocols 10, 442-458 (2015).

Additionally, in another aspect, the invention provides a kit, comprising: (a) the polymerase according to the invention, and optionally, a plurality of different individual nucleotides of the invention and/or packaging materials therefor.

Several Experiments were carried out to show the increased rate of incorporation of nucleotides which have been modified compared to different wildtype polymerases and polymerases of the state of the art. Some of the results are shown in FIGS. 6-8, 10 and 11. Further results with other wildtype polymerases and mutated polymerases from the state of the art also showed an increased rate of incorporation of nucleotides which have been modified as well as an enhanced specificity and sensitivity of the mutated polymerases according to the invention. The polymerases according to the invention show enhanced activity for incorporating bulky nucleotides also when compared to those disclosed in EP 1 664 287 B1.

### FIGURE CAPTIONS

FIG. 1 shows labeled analogs of nucleotides with 3′-O-methylenedisulfide-containing protecting group, where labels are attached to the nucleobase via cleavable oxymethylenedisulfide linker (—OCH2-SS—). The analogs are (clockwise from the top left) for deoxyadenosine, thymidine or deoxyuridine, deoxycytidine and deoxyguanosine.

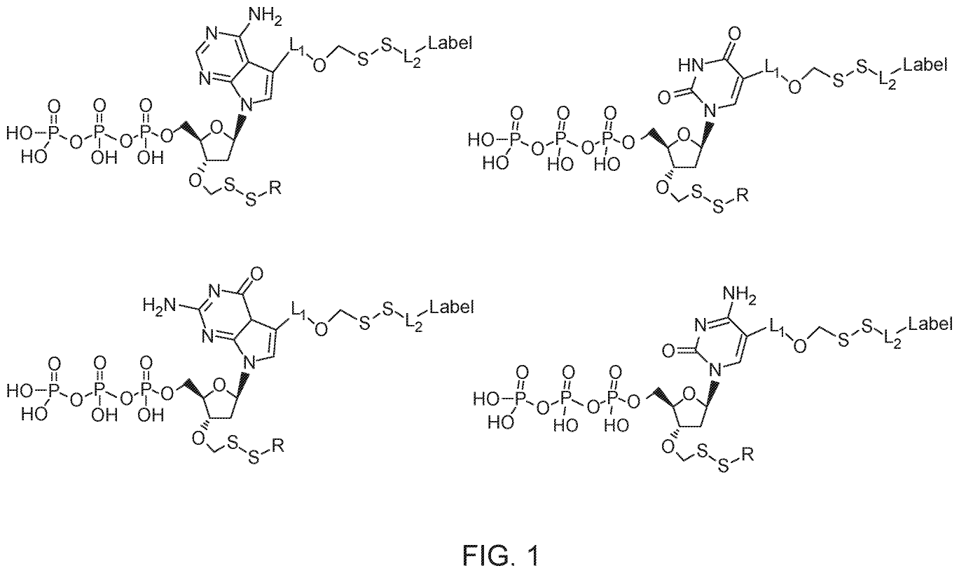

FIG. 2 shows an example of the labeled nucleotides where the spacer of the cleavable linker includes the propargyl ether linker. The analogs are (clockwise from the top left) for deoxyadenosine, thymidine or deoxyuridine, deoxycytidine and deoxyguanosine.

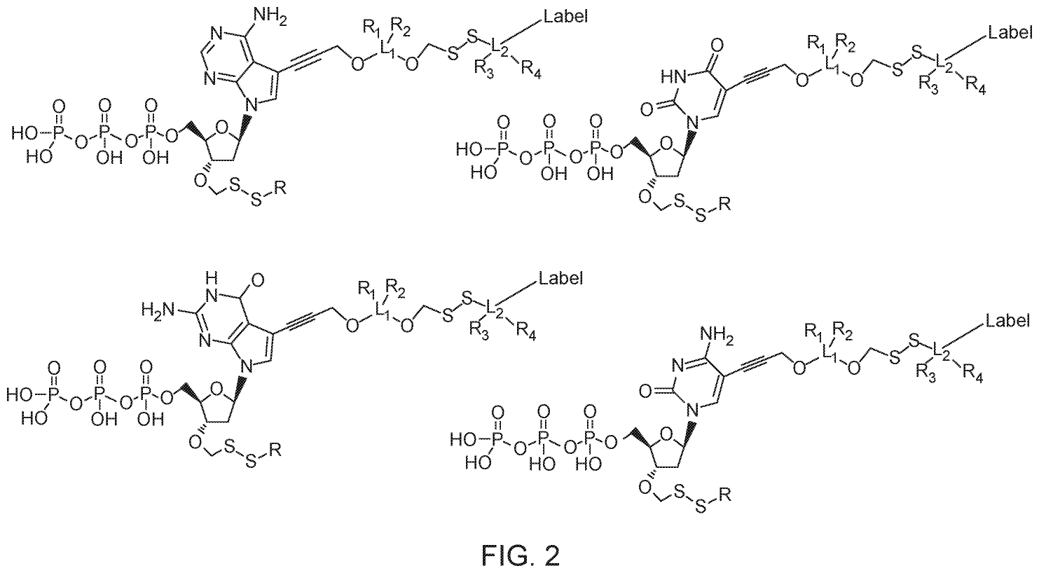

FIG. 3 shows a synthetic route of the labeled nucleotides specific for labeled dT intermediate.

FIG. 4 shows a cleavable linker synthesis starting from an 1,4-butanediol.

FIG. 5 shows the measurement of polymerase performance using extension in solution and capillary electrophoresis. The rate of single base terminating dNTP incorporation is measured. The extended fluorescent primer is detected by capillary electrophoresis (CE). The relative rate of dNTP addition is determined by plots of fraction extended primer over time.

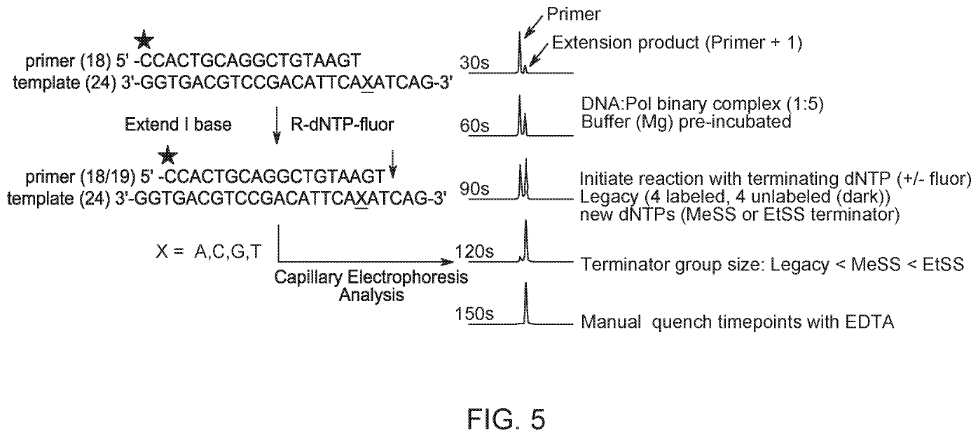

FIG. 6. shows the sequencing performance of Jpol 104 (P. Abyssi construct, SEQ ID No 2) as measured by sequencing KPIs and compared to legacy (T9).

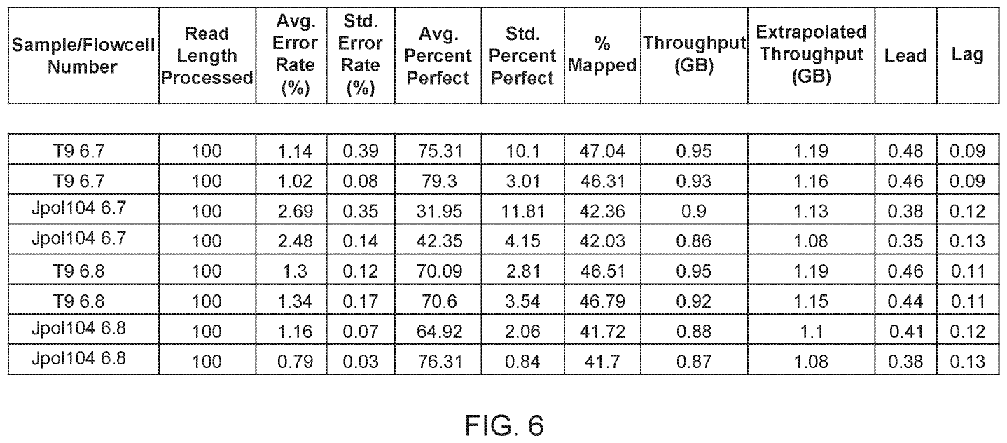

FIG. 7. shows example reads generated by JPol 104 (SEQ ID #2) and T9 polymerases using GR sequencer. The bar chart shows example read as final intensities vs cycle number. The intensities were subjected to background, crosstalk and phasing correction. Color coding is as follows: C—blue, T—green, A—yellow, G—red. For each cycle there is one dominating color indicative of base nucleotide incorporated and base called.

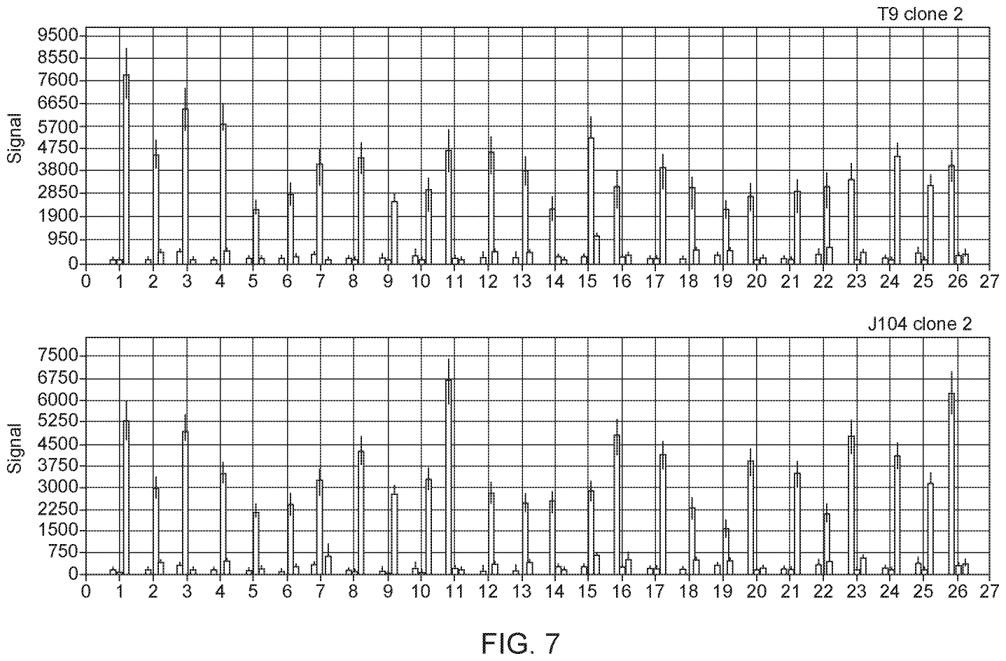

FIG. 8 shows kinetics of incorporation of nucleotide analogs (reversibly terminating dG) as measured by capillary electrophoresis assay. The methodology used here is solution based assay using synthetic DNA template and synthetic primer labeled with fluorophore at 5′end. The template is specific to the nucleotide interrogated. A mixture of pre-annealed primer/template, polymerase and nucleotide are incubated at temperature appropriate for the polymerase studied. After incubation an aliquot is loaded onto capillary electrophoresis system where size separation is performed using denaturing conditions and fluorescence detection. Peaks corresponding to non-extended primer, extended primer and residual nuclease activity (primer degradation) are observed in this trace indicating polymerase ability to incorporate nucleotide analog.

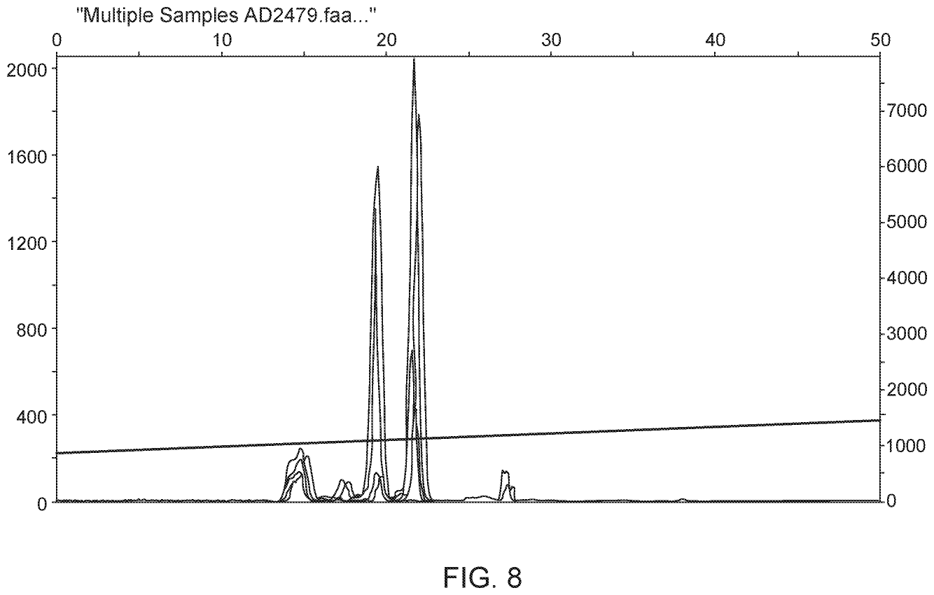

FIG. 9 shows generic universal building blocks structures comprising new cleavable linkers usable with the enzymes of the present invention. PG=Protective Group, LI, L2—linkers (aliphatic, aromatic, mixed polarity straight chain or branched). RG=Reactive Group. In one embodiment of present invention such building blocks carry an Fmoc protective group on one end of the linker and reactive NHS carbonate or carbamate on the other end. This preferred combination is particularly useful in modified nucleotides synthesis comprising new cleavable linkers. A protective group should be removable under conditions compatible with nucleic acid/nucleotides chemistry and the reactive group should be selective. After reaction of the active NHS group on the linker with amine terminating nucleotide, an Fmoc group can be easily removed using base such as piperidine or ammonia, therefore exposing amine group at the terminal end of the linker for the attachment of cleavable marker. A library of compounds comprising variety of markers can be constructed this way very quickly.

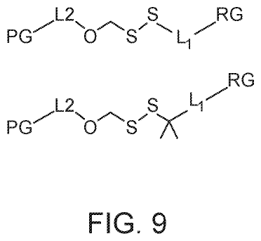

FIG. 10 shows activity of several enzymes of the present invention with either 3′-O—CH₂N₃ or 3′-O—CH₂SSCH₃ terminating groups as measured by capillary electrophoresis assay. Activity is expressed as fraction of extended template over specific time.

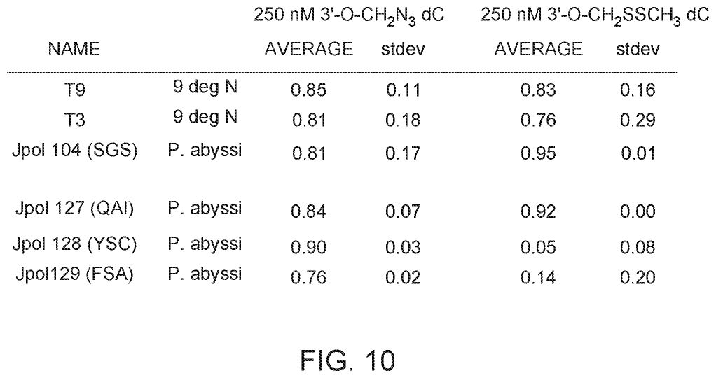

FIG. 11 shows incorporation of fluorescently labeled, reversibly terminating nucleotide Alexa488-dC-3′-O—CH₂SSCH₃ as measured by fluorescence plate based assay for polymerases of the present invention: JPo1104 (SEQ ID #2), JPo1127 (SEQ ID #3), JPo1128 (SEQ ID #4), JPo1129 (SEQ ID #5). Duplex DNA was immobilized on the plate, a solution of polymerase and nucleotide was added and after incubation plate was washed and read with fluorescence plate reader (exc. 490 nm/em. 520 nm).

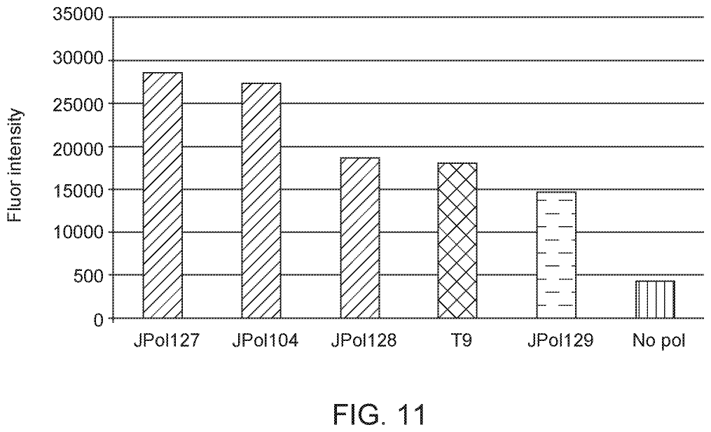

### EXAMPLES

Enzyme Sequences

| | |
|---|---|
| SEQ ID NO.1 | MIIDADYITEDGKPIIRIFKKEKGEFKVEYDRTFRPYIYALLKD |
| gi\|1495740\|emb\| | DSAIDEVKKITAERHGKIVRITEVEKVQKKFLGRPIEVWKLYL |
| CAA90888.1\| | EHPQDVPAIREKIREHPAVVDIFEYDIPFAKRYLIDKGLTPMEG |
| Wild type | NEELTFLAVDIETLYHEGEEFGKGPIIMISYADEEGAKVITWKS |
| DNA-dependent | IDLPYVEVVSSEREMIKRLVKVIREKDPDVIITYNGDNFDFPYL |
| DNA polymerase | LKRAEKLGIKLPLGRDNSEPKMQRMGDSLAVEIKGRIHFDLFP |
| Pyrococcus abyssi | AIRRTINLPTYTLETVYEVIFGKSKEKVYAHEIAEAWETGKGL |
| | ERVAKYSMEDAKVTSELGKEFFPMEAQLARLVGHPVWDVSR |
| | SSTGNLVEWFLLTKAYERNELAPNKPDEREYERRLRESYEGG |
| | YVNEPEKGLWEGIVSLDFRSLYPSIIITHNVSPDTLNRENCKEY |
| | DVAPQVGHRFCKDFPGFIPSLLGNLLEERQKIKKRMKESKDP |
| | VEKKLLDYRQRAIKILANSYYGYYGYAKARWYCKECAESVT |
| | AWGRQYIDLVRRELESRGFKVLYIDTDGLYATIPGAKHEEIKE |
| | KALKFVEYINSKLPGLLELEYEGFYARGFFVTKKKYALIDEEG |
| | KIVTRGLEIVRRDWSEIAKETQAKVLEAILKHGNVDEAVKIVK |
| | EVTEKLSKYEIPPEKLVIYEQITRPLSEYKAIGPHVAVAKRLAA |
| | KGVKVKPGMVIGYIVLXGDGPISKRAIAIEEFDPKKHKYDAE |
| | YYIENQVLPAVERILRAFGYRKEDLRYQKTKQVGLGAWLKF |
| SEQ ID NO. 2 | MIIDADYITEDGKPIIRIFKKEKGEFKVEYDRTFRPYIYALLKD |
| >JPol104_abyssi | DSAIDEVKKITAERHGKIVRITEVEKVQKKFLGRPIEVWKLYL |
| (SGS) | EHPQDVPAIREKIREHPAVVDIFEYDIPFAKRYLIDKGLTPMEG |
| | NEELTFLAVAIATLYHEGEEFGKGPIIMISYADEEGAKVITWK |
| | SIDLPYVEVVSSEREMIKRLVKVIREKDPDVIITYNGDNFDFPY |
| | LLKRAEKLGIKLPLGRDNSEPKMQRMGDSLAVEIKGRIHFDLF |
| | PAIRRTINLPTYTLETVYEVIFGKSKEKVYAHEIAEAWETGKG |
| | LERVAKYSMEDAKVTSELGKEFFPMEAQLARLVGHPVWDVS |
| | RSSTGNLVEWFLLTKAYERNELAPNKPDEREYERRLRESYEG |
| | GYVNEPEKGLWEGIVSLDFRSSGSSIIITHNVSPDTLNRENCKE |
| | YDVAPQVGHRFCKDFPGFIPSLLGNLLEERQKIKKRMKESKD |
| | PVEKKLLDYRQRLIKILANSYYGYYGYAKARWYCKECAESV |
| | TAWGRQYIDLVRRELESRGFKVLYIDTDGLYATIPGAKHEEIK |
| | EKALKFVEYINSKLPGLLELEYEGFYARGFFVTKKKYALIDEE |
| | GKIVTRGLEIVRRDWSEIAKETQAKVLEAILKHGNVDEAVKIV |
| | KEVTEKLSKYEIPPEKLVIYEQITRPLSEYKAIGPHVAVAKRLA |
| | AKGVKVKPGMVIGYIVLRGDGPISKRAIAIEEFDPKKHKYDAE |
| | YYIENQVLPAVERILRAFGYRKEDLRYQKTKQVGLGAWLKF |
| SEQ ID NO. 3 | MIIDADYITEDGKPIIRIFKKEKGEFKVEYDRTFRPYIYALLKD |
| JPol127_QAI_ | DSAIDEVKKITAERHGKIVRITEVEKVQKKFLGRPIEVWKLYL |
| abyssi | EHPQDVPAIREKIREHPAVVDIFEYDIPFAKRYLIDKGLTPMEG |
| (QAI) | NEELTFLAVAIATLYHEGEEFGKGPIIMISYADEEGAKVITWK |
| | SIDLPYVEVVSSEREMIKRLVKVIREKDPDVIITYNGDNFDFPY |
| | LLKRAEKLGIKLPLGRDNSEPKMQRMGDSLAVEIKGRIHFDLF |
| | PAIRRTINLPTYTLETVYEVIFGKSKEKVYAHEIAEAWETGKG |
| | LERVAKYSMEDAKVTSELGKEFFPMEAQLARLVGHPVWDVS |
| | RSSTGNLVEWFLLTKAYERNELAPNKPDEREYERRLRESYEG |
| | GYVNEPEKGLWEGIVSLDFRSQAISIIITHNVSPDTLNRENCKE |
| | YDVAPQVGHRFCKDFPGFIPSLLGNLLEERQKIKKRMKESKD |
| | PVEKKLLDYRQRLIKILANSYYGYYGYAKARWYCKECAESV |
| | TAWGRQYIDLVRRELESRGFKVLYIDTDGLYATIPGAKHEEIK |
| | EKALKFVEYINSKLPGLLELEYEGFYARGFFVTKKKYALIDEE |
| | GKIVTRGLEIVRRDWSEIAKETQAKVLEAILKHGNVDEAVKIV |
| | KEVTEKLSKYEIPPEKLVIYEQITRPLSEYKAIGPHVAVAKRLA |
| | AKGVKVKPGMVIGYIVLRGDGPISKRAIAIEEFDPKKHKYDAE |
| | YYIENQVLPAVERILRAFGYRKEDLRYQKTKQVGLGAWLKFS |
| | GS |
| SEQ ID NO. 4 | MIIDADYITEDGKPIIRIFKKEKGEFKVEYDRTFRPYIYALLKD |
| JPol128_YSC_ | DSAIDEVKKITAERHGKIVRITEVEKVQKKFLGRPIEVWKLYL |
| abyssi | EHPQDVPAIREKIREHPAVVDIFEYDIPFAKRYLIDKGLTPMEG |
| (YSC) | NEELTFLAVAIATLYHEGEEFGKGPIIMISYADEEGAKVITWK |
| | SIDLPYVEVVSSEREMIKRLVKVIREKDPDVIITYNGDNFDFPY |
| | LLKRAEKLGIKLPLGRDNSEPKMQRMGDSLAVEIKGRIHFDLF |
| | PAIRRTINLPTYTLETVYEVIFGKSKEKVYAHEIAEAWETGKG |
| | LERVAKYSMEDAKVTSELGKEFFPMEAQLARLVGHPVWDVS |
| | RSSTGNLVEWFLLTKAYERNELAPNKPDEREYERRLRESYEG |
| | GYVNEPEKGLWEGIVSLDFRSYSCSIIITHNVSPDTLNRENCKE |
| | YDVAPQVGHRFCKDFPGFIPSLLGNLLEERQKIKKRMKESKD |
| | PVEKKLLDYRQRLIKILANSYYGYYGYAKARWYCKECAESV |
| | TAWGRQYIDLVRRELESRGFKVLYIDTDGLYATIPGAKHEEIK |
| | EKALKFVEYINSKLPGLLELEYEGFYARGFFVTKKKYALIDEE |
| | GKIVTRGLEIVRRDWSEIAKETQAKVLEAILKHGNVDEAVKIV |
| | KEVTEKLSKYEIPPEKLVIYEQITRPLSEYKAIGPHVAVAKRLA |
| | AKGVKVKPGMVIGYIVLRGDGPISKRAIAIEEFDPKKHKYDAE |
| | YYIENQVLPAVERILRAFGYRKEDLRYQKTKQVGLGAWLKFS |
| | GS |
| SEQ ID NO. 5 | MIIDADYITEDGKPIIRIFKKEKGEFKVEYDRTFRPYIYALLKD |
| JPol129_FSA_ | DSAIDEVKKITAERHGKIVRITEVEKVQKKFLGRPIEVWKLYL |
| abyssi | EHPQDVPAIREKIREHPAVVDIFEYDIPFAKRYLIDKGLTPMEG |
| (FSA) | NEELTFLAVAIATLYHEGEEFGKGPIIMISYADEEGAKVITWK |
| | SIDLPYVEVVSSEREMIKRLVKVIREKDPDVIITYNGDNFDFPY |
| | LLKRAEKLGIKLPLGRDNSEPKMQRMGDSLAVEIKGRIHFDLF |
| | PAIRRTINLPTYTLETVYEVIFGKSKEKVYAHEIAEAWETGKG |
| | LERVAKYSMEDAKVTSELGKEFFPMEAQLARLVGHPVWDVS |
| | RSSTGNLVEWFLLTKAYERNELAPNKPDEREYERRLRESYEG |
| | GYVNEPEKGLWEGIVSLDFRSFSASIIITHNVSPDTLNRENCKE |
| | YDVAPQVGHRFCKDFPGFIPSLLGNLLEERQKIKKRMKESKD |
| | PVEKKLLDYRQRLIKILANSYYGYYGYAKARWYCKECAESV |
| | TAWGRQYIDLVRRELESRGFKVLYIDTDGLYATIPGAKHEEIK |
| | EKALKFVEYINSKLPGLLELEYEGFYARGFFVTKKKYALIDEE |
| | GKIVTRGLEIVRRDWSEIAKETQAKVLEAILKHGNVDEAVKIV |
| | KEVTEKLSKYEIPPEKLVIYEQITRPLSEYKAIGPHVAVAKRLA |
| | AKGVKVKPGMVIGYIVLRGDGPISKRAIAIEEFDPKKHKYDAE |
| | YYIENQVLPAVERILRAFGYRKEDLRYQKTKQVGLGAWLKFS |
| | GS |

### Example 1

### Synthesis of 3′-O-(methylthiomethyl)-5′-0-(tert-butyldimethylsilyl)-2′-deoxythymidine (2)

5′-0-(tert-butyldimethylsilyl)-2′-deoxythymidine (1) (2.0 g, 5.6 mmol) was dissolved in a mixture consisting of DMSO (10.5 mL), acetic acid (4.8 mL), and acetic anhydride (15.4 mL) in a 250 mL round bottom flask, and stirred for 48 hours at room temperature. The mixture was then quenched by adding saturated K₂CO₃ solution until evolution of gaseous CO2 was stopped. The mixture was then extracted with EtOAc (3×100 mL) using a separating funnel. The combined organic extract was then washed with a saturated solution of NaHCO₃ (2×150 mL) in a partitioning funnel, and the organic layer was dried over Na₂S0₄. The organic part was concentrated by rotary evaporation. The reaction mixture was finally purified by silica gel column chromatography.

### Example 2

### Synthesis of 3′-O-(ethyldithiomethyl)-2′-deoxythymidine (4)

Compound 2 (1.75 g, 4.08 mmol), dried overnight under high vacuum, dissolved in 20 mL dry CH₂Cl₂ was added with EtsN (0.54 mL, 3.87 mmol) and 5.0 g molecular sieve-3A, and stirred for 30 min under Ar atmosphere. The reaction flask was then placed on an ice-bath to bring the temperature to sub-zero, and slowly added with 1.8 eq 1M SO₂CI₂ in CH2CI2 (1.8 mL) and stirred at the same temperature for 1.0 hour. Then the ice-bath was removed to bring the flask to room temperature, and added with a solution of potassium thiotosylate (1.5 g) in 4 mL dry DMF and stirred for 0.5 hour at room temperature.

Then 2 eq EtSH (0.6 mL) was added and stirred additional 40 min. The mixture was then diluted with 50 mL CH₂Cl₂ and filtered through celite-S in a funnel. The sample was washed with adequate amount of CH₂Cl₂ to make sure that the product was filtered out. The CH₂Cl₂ extract was then concentrated and purified by chromatography on a silica gel column (Hex:EtOAC/1:1 to 1:3, Rf=0.3 in Hex:EtOAc/1:1). The resulting crude product was then treated with 2.2 g of NH₄F in 20 mL MeOH. After 36 hours, the reaction was quenched with 20 mL saturated NaHCO₃ and extracted with CH₂Cl₂ by partitioning. The CH₂Cl₂ part was dried over Na₂SO₄ and purified by chromatography (Hex:EtOAc/1:1 to 1:2).

### Example 3

### Synthesis of the triphosphate of 3′-O-(ethyldithiomethyl)-2′-deoxythymidine (5)

In a 25 mL flask, compound 4 (0.268 g, 0.769 mmol) was added with proton sponge (210 mg), equipped with rubber septum. The sample was dried under high vacuum for overnight. The material was then dissolved in 2.6 mL (MeO)₃PO under argon atmosphere. The flask, equipped with Ar-gas supply, was then placed on an ice-bath, stirred to bring the temperature to sub-zero. Then 1.5 equivalents of POCI₃ was added at once by a syringe and stirred at the same temperature for 2 hours under Argon atmosphere. Then the ice-bath was removed and a mixture consisting of tributylammonium-pyrophosphate (1.6 g) and Bu₃N (1.45 mL) in dry DMF (6 mL) was prepared. The entire mixture was added at once and stirred for 10 min. The reaction mixture was then diluted with TEAB buffer (30 mL, 100 mM) and stirred for additional 3 hours at room temperature. The crude product was concentrated by rotary evaporation, and purified by CI 8 Prep HPLC (method: 0 to 5 min 100% A followed by gradient up to 50% B over 72 min, A=50 mM TEAB and B=acetonitrile). After freeze drying of the target fractions, the semi-pure product was further purified by ion exchange HPLC using PL-SAX Prep column (Method: 0 to 5 min 100% A, then gradient up to 70% B over 70 min, where A=15% acetonitrile in water, B=0.85M TEAB buffer in 15% acetonitrile). Final purification was carried out by CI8 Prep HPLC as described above resulting in ˜25% yield of compound 5.

### Example 4

### Synthesis of N⁴-Benzoyl-5′-0-(tert-butyldimethylsilyl)-3′-O-(methylthiomethyl)-2 ‘ deoxycytidine (7)

N⁴-benzoyl-5′-O-(tert-butyldimethylsilyl)-2’-deoxycytidine (6) (50 g, 112.2 mmol) was dissolved in DMSO (210 mL) in a 2 L round bottom flask. It was added sequentially with acetic acid (210 mL) and acetic anhydride (96 mL), and stirred for 48 h at room temperature. During this period of time, a complete conversion to product was observed by TLC (Rf=0.6, EtOAc:hex/10:1 for the product).

The mixture was separated into two equal fractions, and each was transferred to a 2000 mL beaker and neutralized by slowly adding saturated K₂CO₃ solution until CO₂ gas evolution was stopped (pH 8). The mixture was then extracted with EtOAc in a separating funnel. The organic part was then washed with saturated solution of NaHCO₃ (2×1 L) followed by with distilled water (2×1 L), then the organic part was dried over Na₂SO₄.

The organic part was then concentrated by rotary evaporation. The product was then purified by silica gel flash-column chromatography using puriflash column (Hex:EtOAc/1:4 to 1:9, 3 column runs, on 15 um, HC 300 g puriflash column) to obtain N⁴-benzoyl-5′-O-(tert-butyldimethylsilyl)-3′-O-(methylthiomethyl)-2′-deoxycytidine (7) as grey powder in 60% yield.

### Example 5

### N⁴-Benzoyl-3 ‘-0-(ethyldithiomethyl)-5’-0-(tert-butyldimethylsilyl)-2′-deoxycytidine (8)

N⁴-Benzoyl-5′-0-(tert-butyldimethylsilyl)-3′-O-(methylthiomethyl)-2′-deoxycytidine (7) (2.526 g, 5.0 mmol) dissolved in dry CH₂Cl₂ (35 mL) was added with molecular sieve-3A (10 g). The mixture was stirred for 30 minutes. It was then added with Et3N (5.5 mmol), and stirred for 20 minutes on an ice-salt-water bath. It was then added slowly with 1M SO₂CI₂ in CH₂Cl₂ (7.5 mL, 7.5 mmol) using a syringe and stirred at the same temperature for 2 hours under N2-atmosphere. Then benzenethiosulfonic acid sodium salt (1.6 g, 8.0 mmol) in 8 mL dry DMF was added and stirred for 30 minutes at room temperature. Finally, EtSH was added (0.74 mL) and stirred additional 50 minutes at room temperature. The reaction mixture was filtered through celite-S, and washed the product out with CH₂Cl₂. After concentrating the resulting CH₂Cl₂ part, it was purified by flash chromatography using a silica gel column (1:1 to 3:7/Hex:EtOAc) to obtain compound 8 in 54.4% yield.

### Example 6

### N⁴-Benzoyl-3′-O-(ethyldithiomethyl)-2′-deoxycytidine (9)

N⁴-Benzoyl-3 ‘-O-(ethyldithiomethyl)-5′-O-(tert-butyldimethylsilyl)-2’-deoxycytidine (8, 1.50 g, 2.72 mmol) was dissolved in 50 mL THF. Then 1M TBAF in THF (3.3 mL) was added at ice-cold temperature under nitrogen atmosphere. The mixture was stirred for 1 hour at room temperature. Then the reaction was quenched by adding 1 mL MeOH, and solvent was removed after 10 minutes by rotary evaporation. The product was purified by silica gel flash chromatography using gradient 1:1 to 1:9/Hex:EtOAc to result in compound 9. Finally, the synthesis of compound 10 was achieved from compound 9 following the standard synthetic protocol described in the synthesis of compound 5.

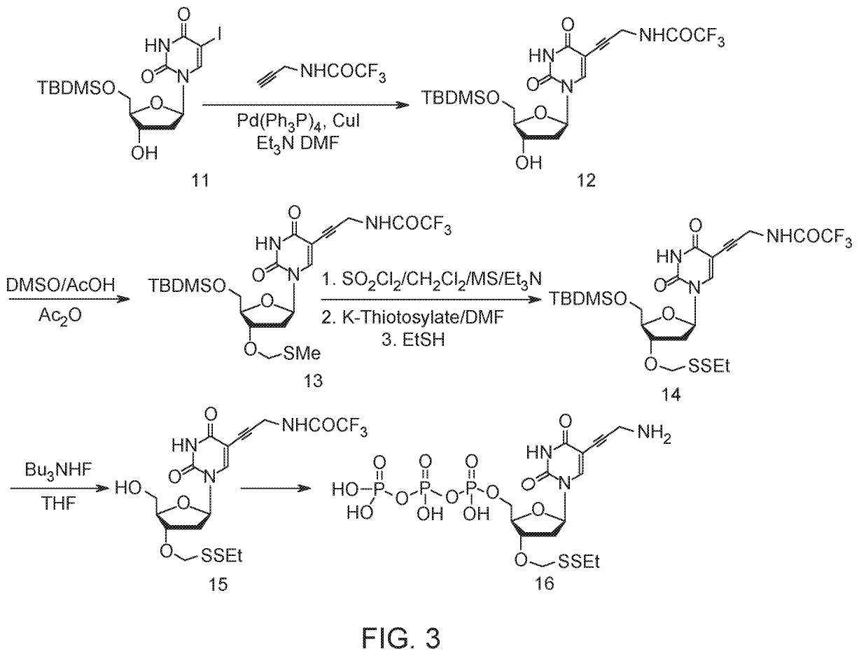

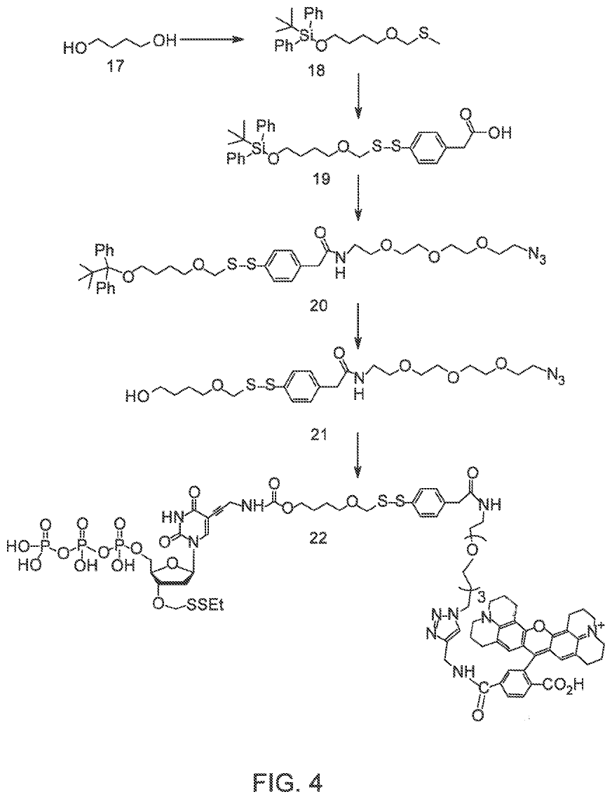

The synthesis of the labeled nucleotides can be achieved following the synthetic routes shown in FIG. 3 and FIG. 4. FIG. 3 is specific for the synthesis of labeled dT intermediate, and other analogs could be synthesized similarly.

1. A polymerase enzyme according to SEQ ID NO. 1 or any polymerase that shares at least 70%, 80%, 85%, 90%, 95%, 98% amino acid sequence identity thereto, comprising the following mutation(s):
a. at position 409 of SEQ ID NO. 1:
i. serine (S) (L409S) or,
ii. glutamine (Q) (L409Q) or,
iii. tyrosine (Y) (L409Y) or,
iv. phenylalanine (F) (L409F)
b. at position 410 of SEQ ID NO. 1:
i. glycine (G) (Y410G) or,
ii. adenine (A) (Y409A) or,
iii. serine (S) (Y409S),
c. at position 411 of SEQ ID NO. 1:
i. serine (S) (P411S) or,
ii. isoleucine (I) (P411I) or,
iii. cysteine (C) (P411C) or,
iv. adenine (A) (P411A),
wherein the enzyme has little or no 3′-5′ exonuclease activity.

2. The polymerase enzyme of claim 1, wherein the polymerase is from an organism belonging to the family of Thermococcaceae, preferably from the genera of Pyrococcus.

3. The polymerase enzyme according to claim 1, wherein the polymerase enzyme comprises a L409S mutation, a Y410G mutation and a P411S mutation; and optionally comprises one or more a D141A mutation, a E143A mutation, or a A486L mutation.

4. The polymerase enzyme according to claim 3, wherein the polymerase enzyme further comprises the A486L mutation.

5. The polymerase enzyme according to claim 1, wherein the polymerase enzyme shares 95% or 98% sequence identity with SEQ ID NO. 1 and comprises the following mutations:
(i) L409S, Y410G, P411S and
(ii) A486L.

6. The polymerase enzyme according to claim 1, wherein the polymerase enzyme exhibits an increased rate of incorporation of nucleotides which have been modified at the 3′ sugar hydroxyl such that the substituent is larger in size than the naturally occurring 3′ hydroxyl group, compared to the control polymerase.

7. A nucleic acid molecule encoding a polymerase enzyme according to claim 1.

8. An expression vector comprising the nucleic acid molecule of claim 7.

9. A method for incorporating nucleotides which have been modified at the 3′ sugar hydroxyl such that the substituent is larger in size than the naturally occurring 3′ hydroxyl group into DNA comprising the following substances (i) a polymerase enzyme according to claim 1, (ii) template DNA, (iii) one or more nucleotides, which have been modified at the 3′ sugar hydroxyl such that the substituent is larger in size than the naturally occurring 3′ hydroxyl group.

10. Use of a polymerase enzyme according to claim 1 for DNA sequencing, DNA labeling, primer extension, amplification or the like.

11. A kit comprising a polymerase enzyme according to claim 1.
